(19) United States
(12) Patent Application Publication
(10) Pub. No.: US 2023/0020383 A1
(43) Pub. Date: Jan. 19, 2023

(54) Footwear, Feet and Animal Paw Disinfecting Doormat

(71) Applicant: Larry Ford, Bronx, NY (US)

(72) Inventor: Larry Ford, Bronx, NY (US)

(21) Appl. No.: 17/868,356

(22) Filed: Jul. 19, 2022

Related U.S. Application Data
(60) Provisional application No. 63/223,227, filed on Jul. 19, 2021.

(51) Int. Cl. A61L 2/10; A47L 23/26

(52) U.S. Cl. CPC A61L 2/10; A47L 23/266; A61L 2202/11; A61L 2202/14

(57) ABSTRACT

The present invention relates to a novel sanitizing device for the bottom of footwear, feet, and animal paws to kill up to 99% of pathogens. The sanitizing device is designed as a doormat and comes in a variety of sizes to accommodate different sized doorways. The doormat is durable, weather-resistant, and chemical free and uses Far-UVC lights for disinfection. The doormat includes a pressure sensor for sensing pressure on a platform of the doormat to activate Far-UVC lights for disinfection and the UVC light is emitted for a preconfigured time period for providing effective disinfection to the footwear, feet and paws placed thereon.

### CROSS-REFERENCE TO RELATED APPLICATION

The present application claims priority to, and the benefit of, U.S. Provisional Application No. 63/223,227, which was filed on Jul. 19, 2021 and is incorporated herein by reference in its entirety.

### FIELD OF THE INVENTION

The present invention relates generally to the field of doormats. More specifically, the present invention relates to a novel sanitizing doormat for disinfecting bottom of footwear, feet, and animal paws using Far-UVC light. The doormat has Far-UVC light sources for emitting Far-UVC light towards a platform of the doormat on which a user or animal stand. The light sources are automatically activated when a user or animal stand on the platform and emit Far-UVC light for a predetermined duration. The doormat eradicates up to 99% pathogens and can be used in homes, offices, schools, hospitals, hotels, airports, fitness centers, and more. Accordingly, the present disclosure makes specific reference thereto. Nonetheless, it is to be appreciated that aspects of the present invention are also equally applicable to other like applications, devices, and methods of manufacture.

### BACKGROUND

By way of background, the greatest source of multiple contamination agents introduced to interiors of homes, hospitals, schools, and offices is carried on contaminated bottoms, of footwear. For providing protection from such contamination, conventional doormats are used. A doormat is a flat object, generally rectangular or oval, usually placed immediately outside or inside the entrance to a house or other building, to allow people to easily scrub, wipe, and/or slide the soles of their shoes on the doormat before entering. A doormat is sometimes also known as a welcome mat, as its location at an entrance constitutes a “welcome” to visitors, and may therefore also bear some word, message, or sign of greeting. Without use of a conventional doormat, people often track dirt, germs, viruses, and bacteria into a home or building via their shoes (i.e. bottom of shoes). This may put other people at risk for contracting illnesses. Many contaminants are brought into a home or business on the bottoms of feet, shoes, and animal paws.

Conventional doormats are used for only removing dirt and debris by scrubbing or wiping their footwear thereon by users. Footwear of users, and paws and legs of animals, carry pathogens and microorganisms that require a more thorough cleaning and disinfection. Conventional doormats do not provide the capability of disinfection of footwear. Further, other methods such as chemical and steam cleaning are difficult to deploy and are also ineffective. Chemicals can also be spread on floors which may prove hazardous. People desire a way of providing effective cleaning and sanitization using the doormats so as to provide effective protection from contamination.

Methods such as hand wash, use of sanitizers and others are available for hand sanitization, but a similar method for effective killing of pathogens from the bottom of feet, footwear, and animal paws is heretofore not known.

Therefore, there exists a long felt need in the art for a doormat apparatus that can disinfect and sanitize footwear, feet, paws, and others placed on the doormat. There is also a long felt need in the art for a doormat that can eradicate up to 99% of pathogens. Additionally, there is a long felt need in the art for a doormat that can be used in homes, offices, hospitals, airports, and more. Moreover, there is a long felt need in the art for a doormat apparatus that provides a chemical free treatment for feet, paws, and footwear to kill pathogens. Further, there is a long felt need in the art for a doormat that eliminates the risk for contracting illnesses due to contamination caused by dirty and contaminated footwear, feet, and paws. Finally, there is a long felt need in the art for an improved doormat that effectively kills pathogens from the bottom of feet, footwear, and animal paws and ensures cleanliness and eradication of contaminants.

The subject matter disclosed and claimed herein, in one embodiment thereof, comprises a footwear sanitizing doormat device. The doormat device includes a housing, a platform supported by the housing and circumscribed by a nylon border. The platform can be configured to support footwear, feet, and paws placed thereon. The platform is further configured to permit transmission of Far-UVC therethrough, a pressure sensor for detecting pressure on the platform, a source of Far-UVC light disposed within the housing for emitting Far-UVC light, and a timer for starting a countdown timer for which the source of Far-UVC light emits the Far-UVC light wherein the source of Far-UVC light is automatically activated when pressure is detected by the pressure sensor.

In this manner, the sanitizing doormat of the present invention accomplishes all of the forgoing objectives and provides users with a doormat designed with integrated UV lights to sanitize shoes, feet, and paws before entering a home or building, and offers a way to ensure cleanliness by eliminating germs viruses, and fungi at the door. The UV light eradicates up to 99% of contaminants and automatically stops emitting UV light after a threshold or predeterminable time period.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a footwear sanitizing doormat device. The footwear sanitizing doormat device is configured to sanitize shoes, feet, and paws, and offers a way to ensure cleanliness by eliminating germs, viruses, and fungi at the door. The device further comprising a generally rectangular housing, a platform supported by the housing and enclosed by a nylon border, wherein the platform is configured to support shoes, feet, and paws placed thereon. The platform is further configured to permit transmission of ultraviolet light therethrough, a pressure sensor for detecting pressure on the platform, a source of ultraviolet light disposed within the housing for emitting ultraviolet light, and a timer for starting a countdown timer for which the source of ultraviolet light emits the ultraviolet light wherein the source of ultraviolet light is automatically activated when pressure is detected by the pressure sensor.

In yet another embodiment, the platform is made from a transparent or translucent material.

In yet another embodiment, the platform is made of one or more from transparent glass, translucent glass, fused silica, or quartz wherein the glass is slightly flexible.

In yet another embodiment, a sanitizing device for shoes, feet, and paws is disclosed. The sanitizing device comprising a housing, an ultraviolet light source adapted to emit ultraviolet light, a platform supported by the housing on which shoes, feet, and paws are placed, wherein the platform includes a plurality of sections for placing the shoes, feet, and paws thereon. The ultraviolet light source is designed to emit ultraviolet light towards the sections and is placed along the nylon border wherein the ultraviolet light source is automatically activated when shoes, feet, or paws are placed on one or more of the sections and is deactivated after a preconfigured time period by a timer.

In yet another embodiment, the sanitizing device includes a built-in power source for providing power to the ultraviolet light source.

In yet another embodiment, the platform includes a discontinuous ultraviolet protective layer preventing ultraviolet light to pass through the platform wherein the layer is not present underneath the sections thereby allowing ultraviolet to pass for sanitizing shoes, feet, and paws placed on one or more of the sections.

In yet another embodiment, the ultraviolet light source is provided power using an external power supply.

In yet another embodiment, the ultraviolet light source provides a Far-UVC light in the range from about 207 nm to about 222 nm.

In yet another embodiment of the present invention, an apparatus for disinfecting feet and shoes of a person and paws of an animal is disclosed. The apparatus having a platform surrounded by a nylon border, the platform is configured to allow germicidal ultraviolet light to pass through to reach feet, shoes, paws placed on a top surface of the platform, a germicidal ultraviolet light source for emitting germicidal ultraviolet light through the platform, a power supply for providing power to the germicidal ultraviolet light source, a timer for automatically deactivating the germicidal ultraviolet light source and a sensor for automatically activating the germicidal ultraviolet light source when feet, shoes, or paws are detected on the platform.

In yet another embodiment, the sensor is a pressure sensor and is coupled to the platform.

In yet another embodiment, the apparatus is configured to be cleaned using a chemical free cleaner.

In yet another embodiment, the apparatus is flexible and washable.

In yet another embodiment, the apparatus includes a generally rectangular, square, oval, or circular shape.

In yet another embodiment, the platform includes a specific area through which the germicidal ultraviolet light is passed therethrough.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of a sanitizing doormat of the present invention in accordance with the disclosed architecture;

FIG. 2 illustrates a perspective view showing a user standing on the sanitizing doormat of FIG. 1 for sanitization of footwear in accordance with the disclosed architecture;

FIG. 3 illustrates perspective view of another embodiment of the sanitizing doormat of the present invention in accordance with the disclosed architecture;

FIG. 4 illustrates a perspective view showing a dog standing on the doormat of FIG. 3 of the present invention in accordance with the disclosed architecture;

FIG. 5 illustrates various shapes and designs of various embodiments of the sanitizing doormat of various embodiments of the present invention in in accordance with the disclosed architecture; and

FIG. 6 illustrates a flow diagram showing exemplary steps performed during use of the doormat of the present invention in activating the ultraviolet light source in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long felt need in the art for a doormat apparatus that can disinfect and sanitize footwear, feet, paws, and others placed on the doormat. There is also a long felt need in the art for a doormat that can eradicate up to 99% of pathogens. Additionally, there is a long felt need in the art for a doormat that can be used in homes, offices, hospitals, airports, and more. Moreover, there is a long felt need in the art for a doormat apparatus that provides a chemical free treatment for feet, paws, and footwear to kill pathogens. Further, there is a long felt need in the art for a doormat that eliminates the risk for contracting illnesses due to contamination caused by dirty and contaminated footwear, feet, and paws. Finally, there is a long felt need in the art for an improved doormat that effectively kills pathogens from the bottom of feet, footwear, and animal paws, and ensures cleanliness and eradication of contaminants.

The present invention, in one exemplary embodiment, is a novel sanitizing device for disinfecting bottom of shoes, feet, and animal paws. The sanitizing device comprising a housing, an ultraviolet light source adapted to emit Far-UVC ultraviolet light, a platform supported by the housing on which shoes, feet, and paws are placed, the platform having a plurality of sections for placing the shoes, feet, and paws thereon, the ultraviolet light source is designed to emit Far-UVC ultraviolet light towards the sections and is placed along the nylon border wherein the ultraviolet light source is automatically activated when shoes, feet, or paws are placed on one or more of the sections and can be autonomously deactivated after a preconfigured time period by a timer.

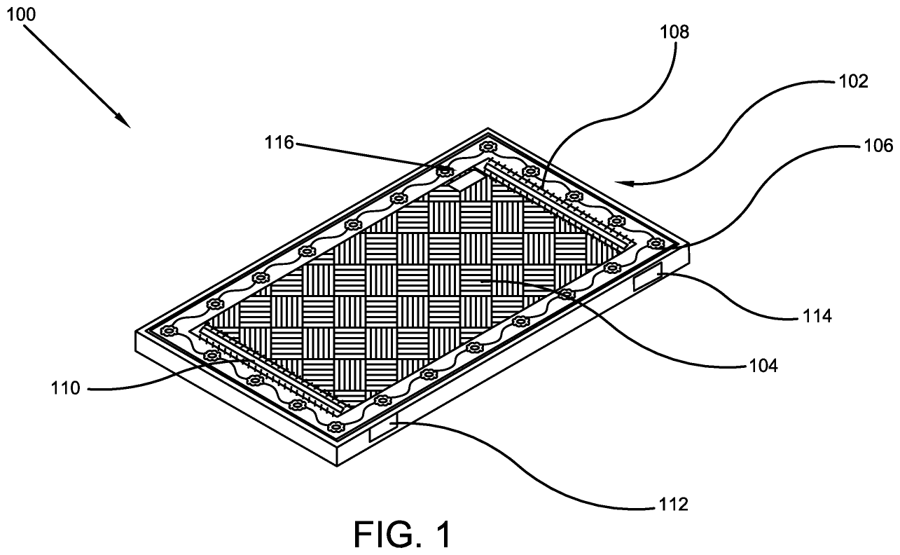

Referring initially to the drawings, FIG. 1 illustrates a perspective view of one potential embodiment of a sanitizing doormat 100 of the present invention in accordance with the disclosed architecture. The sanitizing doormat 100 of the present invention is designed as a durable, weather-resistant, and chemical free doormat that can be stepped onto for activating germicidal ultraviolet light for sanitization of shoes, feet, and paws. More specifically, the doormat 100 includes a housing 102 that is of a general rectangular shape in the present embodiment. The housing 102 includes a platform 104 on which a user or an animal stand for the shoes, feet, or paws to get disinfected. The platform 104 is surrounded by a decorative nylon border 106 that forms the part of the housing 102. The platform 104 can be integrated into the housing 102 and can also be removably attached to the housing 102 in accordance with the present disclosure.

The platform 104 is preferably translucent but can be transparent or opaque based on preference of users and manufacturing designs. For providing sanitization, the doormat apparatus 100 has one or more germicidal ultraviolet light sources 108,110 in the form of ultraviolet light lamps and are positioned inside the housing 102 and preferably along the border 106. The germicidal ultraviolet light sources 108,110 are configured to emit germicidal ultraviolet light (UV light) towards the platform 104 such that the platform 104 allows the UV light to pass through thereby enabling sanitization of the feet, shoes, or paws placed on the platform 104. The germicidal ultraviolet light sources 108,110 can have optional reflectors for reflecting the UV light towards the platform 104. The light sources 108,110 can be mounted proximal to opposing ends of the platform 104.

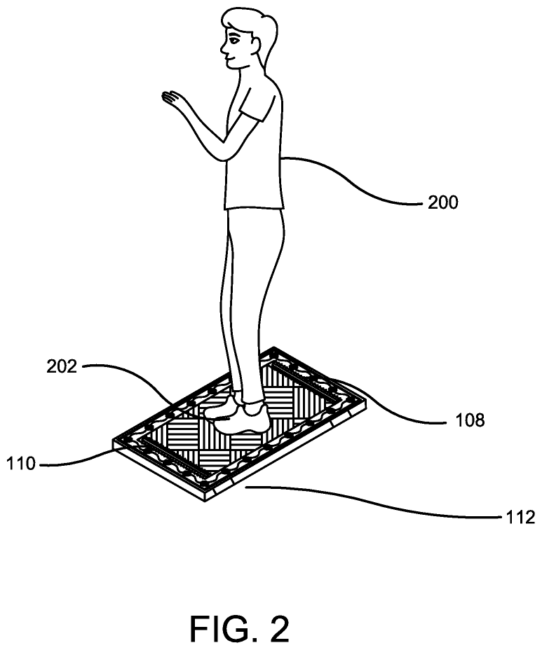

The doormat 100 includes a pressure sensor 112 that is adapted to detect pressure on the platform 104. When pressure is detected on the platform 104 by the pressure sensor 112, then, the germicidal ultraviolet light sources 108,110 are automatically activated for emitting the germicidal UV light towards the platform 104. In the present embodiment, the doormat 100 has integrated and rechargeable battery 114 for providing power to the germicidal ultraviolet light sources 108,110. Alternatively, external power supply may be used for providing power to the germicidal ultraviolet light sources 108,110 as best shown in FIG. 2.

The germicidal ultraviolet light sources 108,110 are configured to emit UV light in the germicidal range (preferably in the Far-UVC range) and having a wavelength in the range from about 207 nm to about 222 nm, and more specifically having a wavelength in the range from about 208 nm to about 220 nm. The germicidal UV light emitted by the germicidal ultraviolet light sources 108,110 deactivates DNA of bacteria, viruses, and other pathogens and thus provides a complete sanitization. Further, the UV light can sanitize the shoes, feet, or paws of animals within 5-10 seconds without causing any hazard to the skin of a user or animal standing on the platform 104.

A timer module 116 positioned inside the housing 102 is configured to activate a countdown timer once the germicidal ultraviolet light sources 108,110 are activated. The use of the timer module 116 autonomously deactivates the germicidal ultraviolet light sources 108,110 when the preconfigured countdown timer period has elapsed. This ensures that the germicidal ultraviolet light sources 108,110 are not unnecessarily activated for extended periods of time and also helps in conserving power.

It should be understood that although the sanitization from pathogens is the main feature and application of the doormat apparatus 100, the platform 104 is also designed to capture dirt and debris from shoes, feet, and paws and can be used to scrub and wipe feet for removing dirt and debris. Further, the platform 104 can withstand the weight or pressure of a heavy person standing on the platform 104. The doormat 100 can also be cleaned with an organic chemical free cleaner for reuse.

FIG. 2 illustrates a perspective view showing a user standing on the sanitizing doormat 100 of FIG. 1 for sanitization of footwear in accordance with the disclosed architecture. As shown, the doormat 100 sanitizes footwear 202 of the user 200 standing on the platform 104. In use, when the user 200 stands on the platform 104, the pressure sensor 112 detects the pressure on the platform 104 and the germicidal ultraviolet light sources 108,110 are automatically activated for transmitting UV-C light towards the platform 104. The platform 104 allows the Far-UVC light to pass through to sanitize the footwear by killing pathogens and other microbes without causing any harm to the standing user 200.

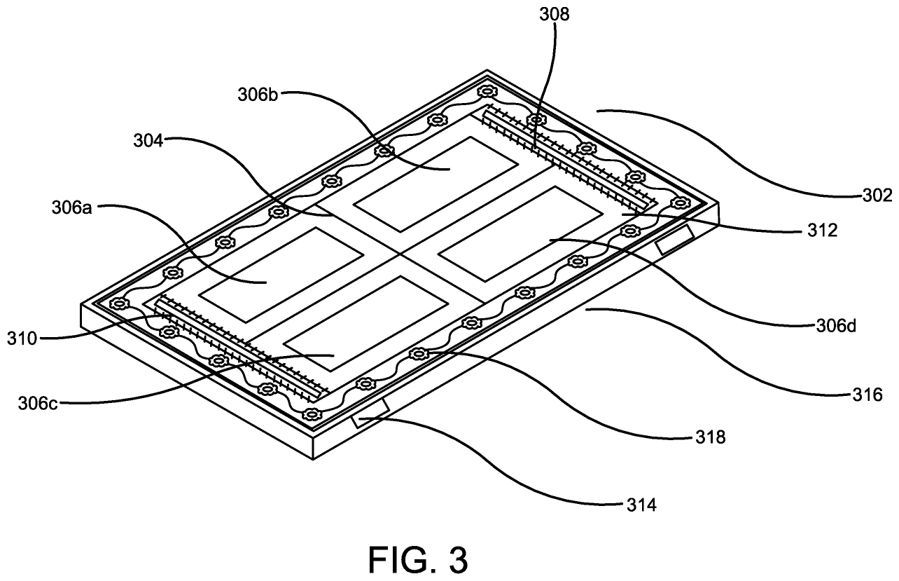

FIG. 3 illustrates a perspective view of another embodiment of the sanitizing doormat of the present invention in accordance with the disclosed architecture. The sanitizing doormat 300 of the present embodiment includes a housing 302 that includes a central platform 304 upon which a user or an animal stands. More specifically, the platform 304 includes a plurality of sections referenced through the numerals 306a,306b,306c,306d on which the feet, paws, or footwear are placed for disinfection. The germicidal ultraviolet light sources 308,310 can be disposed within the housing 302 to emit Far-UVC light towards the platform 304.

The platform 304 includes a discontinuous ultraviolet blocking layer 312 that covers the platform 304 except at the positions underneath the sections 306a-d. This allows transmission of Far-UVC light through the platform 304 only at the sections 306a-d. This embodiment provides more protection to the user and animal standing on the platform 304 from ultraviolet light.

The doormat 300 includes a pressure sensor 314 for detecting pressure on the platform 304 for automatically activating the germicidal ultraviolet light sources 308,310. The doormat 300 includes a power port 316 for receiving power from an external supply for providing power to the germicidal ultraviolet light sources 308,310. Similar to the doormat 100 of FIG. 1, the housing 302 includes a border 318 surrounding the platform 304 for decorative and aesthetic appeal.

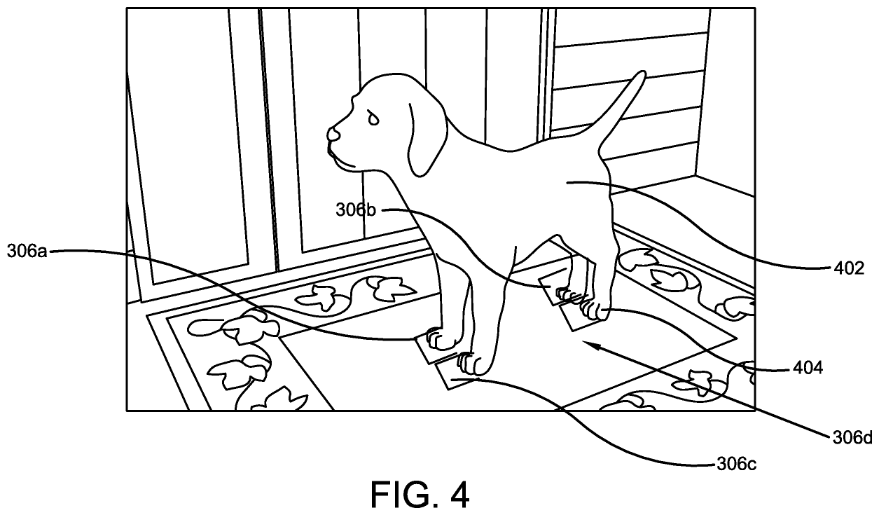

FIG. 4 illustrates a perspective view showing a dog standing on the doormat 300 of FIG. 3 of the present invention in accordance with the disclosed architecture. As shown, a dog 402 standing on the platform 304 has paws 404 on the sections 306a-d. As the pressure on the platform 304 is detected by the pressure monitor 314, the Far-UVC light is emitted and is used for disinfecting the paws 404 of the dog 402. The remaining area of the platform 302 being covered internally with an ultraviolet light blocking layer blocks the Far-UVC light from passing through the platform 304.

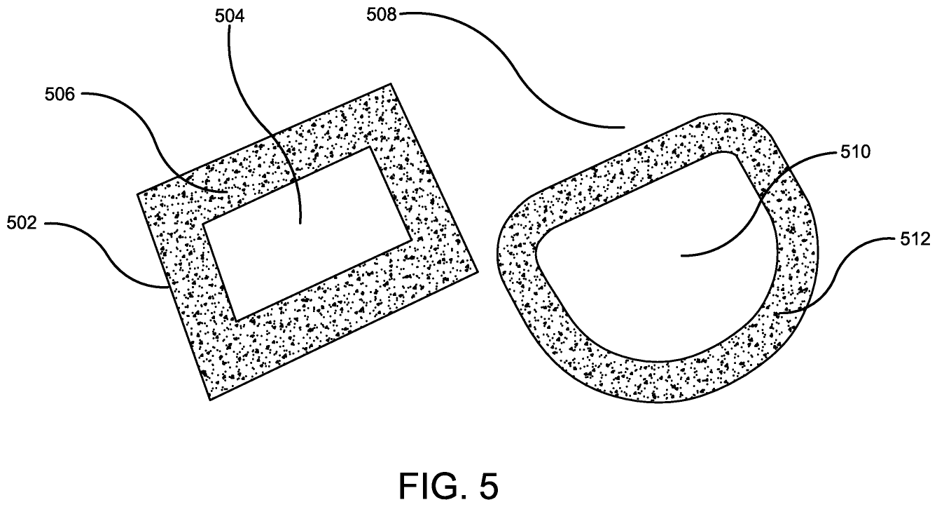

FIG. 5 illustrates various shapes and designs of various embodiments of the sanitizing doormat of various embodiments of the present invention in accordance with the disclosed architecture. As shown, the doormat 502 is a generally square shaped doormat having a rectangular platform 504 and a planar border 506 that may come in a variety of colors.

The D-shaped doormat 508 includes a D-shaped platform 510 on which a user or animal can stand for disinfecting bottom of footwear, feet, or paws and is surrounded by a designer border 512.

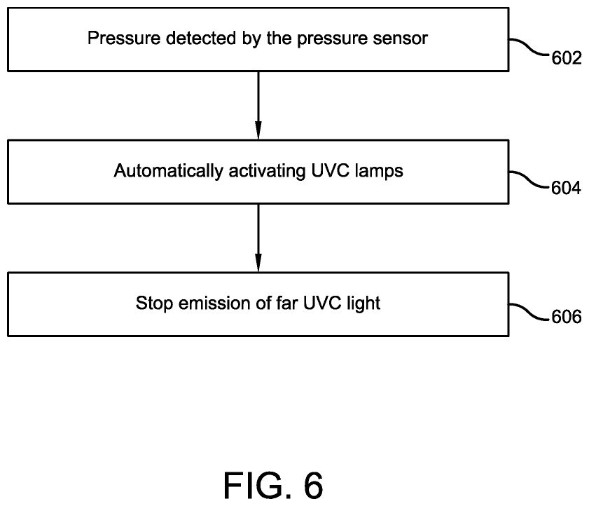

FIG. 6 illustrates a flow diagram showing exemplary steps performed by the doormat 100,300 of the present invention in activating the ultraviolet light source in accordance with the disclosed architecture. As shown, initially, pressure is detected by the pressure sensor of the doormat (Step 602). As soon as the pressure is detected on the platform of the doormat, ultraviolet light source is automatically activated for emitting Far-UVC light towards the platform (Step 604). Thereafter, after the preconfigured time, the Far-UVC light emission is stopped (Step 606).

It should be appreciated that the doormats 100,300 can eradicate up to 99 percent of contaminants and comes in a variety of sizes to accommodate different sized doorways and can be used in homes, offices, schools, hospitals, hotels, airports, fitness centers, government agencies, and more.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “sanitizing doormat”, “footwear sanitizing doormat device”, “device”, “footwear, feet and animal paws disinfecting doormat apparatus”, and “doormat” are interchangeable and refer to the footwear, feet and animal paws disinfecting doormat apparatus 100,300 of the present invention.

Notwithstanding the forgoing, the footwear, feet and animal paws disinfecting doormat apparatus 100,300 of the present invention can be of any suitable size and configuration as is known in the art without affecting the overall concept of the invention, provided that it accomplishes the above stated objectives. One of ordinary skill in the art will appreciate that the size, configuration, and material of the footwear, feet and animal paws disinfecting doormat apparatus 100,300 as shown in the FIGS. are for illustrative purposes only, and that many other sizes and shapes of the footwear, feet and animal paws disinfecting doormat apparatus 100,300 are well within the scope of the present disclosure. Although the dimensions of the footwear, feet and animal paws disinfecting doormat apparatus 100,300 are important design parameters for user convenience, the footwear, feet and animal paws disinfecting doormat apparatus 100,300 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A sanitizing doormat comprising:
a housing comprising a pair of opposing sides and a platform upon which a user or an animal stands;
a plurality of ultraviolet (UV) light sources mounted inside said housing for disinfecting a footwear, feet or paws of the user or the animal standing upon said platform, wherein said plurality of UV light sources are mounted on the pair of opposing sides of said housing and emit a UV light towards said platform; and
a pressure sensor adapted to detect a pressure on said platform, wherein said pressure sensor actuates said plurality of UV light sources when the pressure of the user or the animal standing upon said platform is detected.

2. The sanitizing doormat of claim 1 further comprising a timer module mounted to said housing and configured to activate a countdown timer once said plurality of UV light sources are actuated, wherein said timer module autonomously deactivates said plurality of UV light sources when a preconfigured countdown timer period has elapsed.

3. The sanitizing doormat of claim 2 further comprising a rechargeable battery for providing power to said plurality of UV light sources.

4. The sanitizing doormat of claim 1, wherein said sanitizing doormat comprises a shape selected from a group consisting of a rectangle, a square, an oval and a circle.

5. The sanitizing doormat of claim 1, wherein said platform is selectively removable from said housing.

6. The sanitizing doormat of claim 1, wherein said platform is translucent.

7. The sanitizing doormat of claim 1, wherein said plurality of UV light sources are FAR-UVC lights.

8. The sanitizing doormat of claim 7, wherein said FAR-UVC lights emit a UV light having a wavelength in the range of about 207 nm to about 222 nm.

9. The sanitizing doormat of claim 7, wherein said FAR-UVC lights emit a UV light having a wavelength in the range of about 208 nm to about 220 nm.

10. A sanitizing doormat comprising:
a housing comprising a first side, a second side and a platform upon which a user or an animal stands;
a plurality of FAR-UVC light sources mounted on the first and second sides of the housing for disinfecting a footwear, feet or paws of the user or the animal standing upon said platform, wherein said plurality of FAR-UVC light sources emit a FAR-UVC light towards said platform and further wherein the plurality of FAR-UVC light sources have a wavelength in the range of about 207 nm to about 222 nm; and
a pressure sensor adapted to detect a pressure on said platform, wherein said pressure sensor actuates said plurality of FAR-UVC light sources when the pressure of the user or the animal standing upon said platform is detected.

11. The sanitizing doormat of claim 10 further comprising a timer module mounted to said housing and configured to activate a countdown timer once said plurality of FAR-UVC light sources are actuated, wherein said timer module autonomously deactivates said plurality of FAR-UVC light sources when a preconfigured countdown timer period has elapsed.

12. The sanitizing doormat of claim 10 further comprising a rechargeable battery for providing power to said plurality of FAR-UVC light sources.

13. The sanitizing doormat of claim 10, wherein said sanitizing doormat comprises a shape selected from a group consisting of a rectangle, a square, an oval and a circle.

14. The sanitizing doormat of claim 10, wherein said platform is selectively removable from said housing.

15. The sanitizing doormat of claim 10, wherein said platform is translucent.

16. The sanitizing doormat of claim 10, wherein said plurality of FAR-UVC lights emit a FAR-UVC light having a wavelength in the range from about 208 nm to about 220 nm.

17. A sanitizing doormat comprising:
a housing and a plurality of ultraviolet (UV) light sources mounted inside said housing;
said housing includes a platform upon which a user or an animal stands, wherein said platform includes a plurality of sections;
a UV blocking layer covering said platform around said plurality of sections for limiting transmission of said plurality of UV light sources only at said plurality of sections;
a pressure sensor adapted to detect pressure on said platform;
wherein said pressure sensor actuates said plurality of UV light sources when said pressure of the user or animal standing upon said platform is detected;
wherein said plurality of UV light sources disinfect footwear, feet, or paws of the user or animal standing upon said platform; and
further wherein said plurality of UV light sources are FAR-UVC lights

18. The sanitizing doormat of claim 17 further comprising a timer module mounted to said housing configured to activate a countdown timer once said plurality of UV light sources are actuated, wherein said timer module autonomously deactivates said plurality of UV light sources when a preconfigured countdown timer period has elapsed.

19. The sanitizing doormat of claim 17 further comprising a rechargeable battery for providing power to said plurality of UV light sources.

20. The sanitizing doormat of claim 17, wherein said plurality of UVC lights emit a UV light having a wavelength in the range of about 207 nm to about 222 nm.
